(19) United States
(12) Patent Application Publication
(10) Pub. No.: US 2004/0158940 A1
(43) Pub. Date: Aug. 19, 2004

(54) Phase-separated rinse-off hair coloring/cleansing products

(75) Inventors: Robert Lee Wells, Cincinnati, OH (US); Elizabeth Ann Crane, Martinez, CA (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/774,770

(22) Filed: Feb. 9, 2004

Related U.S. Application Data
(63) Continuation of application No. 09/551,035, filed on Apr. 17, 2000, now abandoned.

(51) Int. Cl. A61K 7/13

(52) U.S. Cl. 8/406

(57) ABSTRACT

Phase-separated rinse-off hair coloring/cleansing compositions comprise water soluble dye materials, surfactant materials, and water, wherein the water soluble dyes are concentrated in a dispersed phase of liquid emulsion droplets dispersed within the droplets to the extent that the intensity of the color imparted by the dye materials to the droplets is greater than the intensity of the color imparted by the dye materials to continuous aqueous phase. The dispersed phase may be formed by the combination of the surfactant materials and the dye materials. Methods of coloring and cleansing hair are also disclosed.

### CROSS REFERENCE TO RELATED APPLICATION

The application is a continuation of application Ser. No. 09/551,035 (Case 8048) filed on Apr. 17, 2000.

### FIELD OF THE INVENTION

The present invention relates to phase-separated rinse-off hair coloring/cleansing compositions and to methods of coloring and cleansing hair therewith.

### BACKGROUND OF THE INVENTION

This invention relates to compositions for imparting color to hair via rinse-off hair cleansing products.

Hair tinting shampoos have been known and have been on the market for some time. They normally contain anionic surfactants, particularly alkyl sulfates and alkyl ether sulfates, and at least one direct hair dye in an aqueous medium. While these shampoos present good lathering properties, the intensity of the hair coloration achieved is often not satisfactory. Although attempts have been made to overcome this disadvantage, optimal coloring results have not been attained. Further, it has been difficult to achieve adequate coloring of gray hair.

Attempts have been made to improve the intensity of hair coloring by using direct dyes in single-phase shampoos which contain anionic and/or amphoteric surfactants, cationic polymers, and solubilizing agents which, upon dilution, form coacervates which increase dye deposition. Other attempts include the following: Adding an organic solvent to prevent formation of a complex in the product of a conventional dye intermediate and a cationic polymer; upon dilution, an insoluble complex assertedly forms and deposits. Other methods include adding anionic and/or amphoteric surfactants to cationic dyes without forming separated liquid phases. More conventional techniques utilize oxidative dye systems with anionic surfactants which deliver a good level of hair color, but also cause hair damage.

It has now been found that coloring actives which are a part of a pre-formed separated liquid phase in the hair dyeing product are efficiently deposited on hair. Formation of such a separated phase during dilution is less efficient because of kinetic factors and the obvious dilution effect, which results in reduced deposition. With water soluble actives, phase separation can be accomplished via coacervation. In addition, it has been found that coacervation of cationic dyes with specific types of surfactants can be achieved in shampoos. In addition, it has now also been found that certain dyes will partition preferentially into separated phases with the surfactant materials of the shampoos to deliver enhanced deposition.

In view of the foregoing, it is an object of this invention to provide a rinse-off hair coloring cleanser which imparts increased color to hair. It is a further object of this invention to deliver color without damaging the hair. It is another object of this invention to provide a method of coloring hair concurrently with a shampooing operation.

### BACKGROUND ART

The following relate to rinse-off hair coloring/cleansing compositions: U.S. Pat. No. 4,93,430, Hefford et. al., Jul. 24, 1990; EP 0089749 A2, Bristol-Myers Co. Sep. 28, 1983; U.S. Pat. No. 5,393,305, Cohen et. al., Feb. 28, 1995; U.S. Pat. No. 5,635,461, Onitsuka et al., Jun. 3, 1997; EP 0819422A1, Kao Corp., Jan. 21, 1998; U.S. Pat. No. 4,168,144, Curry et al., Sep. 18, 1979; U.S. Pat. No. 4,975,092, Chan et al., Dec. 4, 1990; DE 4441359, Kao Corp., May 23, 1996; FR 2096377, Helene Curtis, Feb. 18, 1972; EP 0137178 A2, Beecham Group PLC, Apr. 17, 1985; EP 0146350 A2, Unilever PLC, Jun. 26, 1985; EP 0312343 A3, Unilever PLC, Apr. 19, 1989; U.S. Pat. No. 5,089,257, Schrader et al., Feb. 18, 1992; U.S. Pat. No. 5,376,146, Casperson et al., Dec. 27, 1994; U.S. Pat. No. 3,930,792, Alperin et al., Jan. 6, 1976; U.S. Pat. No. 5,635,461, Onitsuka et al., Jun. 3, 1997; Robbins, C. R., Chemical and Physical Behavior of Human Hair, 2^{nd} Ed., pp. 185-190, Springer-Verlag, New York, 1988; Berthiaume, M. D., Drug & Cosmetic Industry, pp. 42, April, 1997; Zviak, C., The Science of Hair Care, Ch. 7, Marcel Dekker, Inc., New York, 1986; Formulary, Cosmetics & Toiletries, Vol. 113(6), pp. 94, June, 1998; The Arianor Haircare Formula Index, Warner-Jenkinson Europe, Ltd., King's Lynn, Norfolk PE 304JJ.

### SUMMARY OF THE INVENTION

In one embodiment, the present invention relates to phase-separated rinse-off hair coloring/cleansing compositions, comprising:

a.) from about 0.1% to about 10% by weight of water-soluble dye materials;

b.) from about 5% to about 50% by weight of surfactant materials comprising a member selected from the group consisting of anionic surfactant, amphoteric surfactant, and mixtures thereof;

c.) from about 40% to about 94.9% by weight of water;

said water-soluble dye materials being concentrated in a phase of liquid emulsion droplets, said phase being dispersed within a continuous aqueous phase; wherein said dye materials are concentrated within said droplets to the extent that the intensity of the color imparted by the dye materials to the droplets is greater than the intensity of the color imparted by the dye materials to the continuous aqueous phase, the dispersed droplets being formed by the combination of the surfactant materials and the water-soluble dye materials.

In another embodiment, the surfactant materials of the composition set forth above form a separated aqueous phase and the dye materials preferentially partition into this separated aqueous phase. Again, in this embodiment, the dye materials are concentrated in the phase-separated droplets.

The present invention further relates to methods of coloring and cleansing hair with the coloring/cleansing compositions.

### DETAILED DESCRIPTION OF THE INVENTION

The present invention is based on the discovery that improved hair coloring can be achieved using direct dyes that are concentrated in a separated phase or coacervate within an otherwise water-based product. This is achieved by using water-soluble dyes which preferentially partition into and/or reside in the separated phase or coacervate in preference over the continuous phase of the composition.

Theoretically, it may be possible to predict the formation of separated phases and/or coacervates that are useful herein based on the structures of the surfactant and other materials in the systems. However, such theoretical predictions are insufficient in the present circumstances wherein the dye molecules become involved with the surfactant and modify its physical characteristics. Fortunately, however, the selection of proper combinations of dye/surfactant/water can be made without undue experimentation by means of visual observations and by using simple light microscopy. Under microscopic examination of the present compositions at 20° C.-30° C., the dye is seen to be concentrated in droplets having an average size in the range from 0.05 μm to 100 μm in diameter. In preferred compositions, the average size of the droplets ranges from about 0.2 μm to about 50 μm in diameter with no droplets being larger than about 100 μm. Excessively large droplets would lead to uneven coloring of the hair shaft.

In the present compositions, the dye molecules must remain concentrated in the droplets which comprise the dispersed phase or in the coacervate. Accordingly, it is necessary to minimize the migration of the dyes into the aqueous phase. Therefore, the aqueous phase preferably contains no more than about 1% by weight, and most preferably, is substantially free of, water-soluble organic solvents such as methanol, ethanol, isopropanol, butanol, benzyl alcohol, hexylene glycol, carbitol, glycerol, polyols, glycols, other C₃-C₉ solvent alcohols and the like, which are sometimes used in dyeing compositions. Longer-chain alcohols, e.g., cetyl and stearyl, are neither water-soluble nor particularly good solvents, and can be used at levels up to about 7% without disrupting the system.

Non-surfactant electrolytes, such as water soluble inorganic salts, can be used in the present composition to promote the formation of the dye-containing separated phase or coacervate. However, care should be taken not to use excessive amounts of such electrolytes if cationic materials are co-present with the anionic and/or amphoteric surfactants. This is due to the fact that such electrolytes can undesirably promote the water solubility of such systems. In general, the amount of non-surfactant electrolyte in such systems is less than about 10%, preferably less than about 4%.

Moreover, it is to be understood that the appropriate selection of the appropriate dye and the appropriate surfactant will critically impact the formation of useful separated phases and/or coacervates in the product. This is particularly true in circumstances where even minor amounts of solvents, as noted above, may be present. For example, the combination of betaine surfactant with cationic dyes and hexylene glycol are not suitable for use herein. Again, such matters can be determined by microscopic analysis, without undue experimentation.

In addition, certain types of surfactants are not useful herein due to cost considerations, problems associated with eye and skin irritation, hardness sensitivity, low lathering profile and their diminished ability to concentrate the dye molecules in the phase/coacervate droplets. Included among these surfactants are the alkyl phenol ethoxylates, the fatty acid soaps (carboxylates) and the alkyl amidoether carboxylates.

The rinse-off coloring compositions of the present invention comprise the essential ingredients listed above, as well any of the additional or optional ingredients described hereinafter. All percentages, parts and ratios are based on the total weight of the compositions of the present invention, unless otherwise specified. All such weights as they pertain to listed ingredients are based on the active level and, therefore, do not include carriers or by-products that may be included in commercially available materials, unless otherwise specified.

The term, “phase separation” or “separated phases”, as used herein, means the formation of two thermodynamically stable liquid phases which exist, not as distinct bulk layers, but as a stable emulsion comprising droplets of one phase dispersed in another phase.

The term “visually distinct”, as used herein, refers to droplets or droplet phases suspended in a continuous phase such that, microscopically, the droplets or droplet phases are visually separate and distinct from the continuous phase when viewed with the aid of a conventional light microscope under 100× to 620× magnification. Such droplets may be present in the composition as manufactured or may form spontaneously from two separated layers when the composition is shaken by hand (i.e., “shake well before using”) immediately prior to application to hair.

The term “water soluble”, as used herein, refers to any material that is sufficiently soluble in water to form a substantially clear solution to the naked eye at a concentration of at least 0.2% by weight of the material in water at 25° C.

The term “dispersed phase”, as used herein, refers to discrete particles of a composition different from that of the continuous phase. The continuous phase is the phase in which any point may be reached from any other point without passing through another phase.

The term “aqueous phase”, as used herein, refers to a phase in which water is the primary solvent.

The term “liquid crystal phase”, as used herein, refers to a phase which is ordered along only one or two orthogonal directions and disordered along the other directions.

The term “coacervation”, as used herein, refers to the separation of a solution into two immiscible liquid phases; a dense coacervate phase which is relatively concentrated in the species causing separation and a dilute equilibrium phase. Coacervate formation is dependent upon a variety of criteria such as molecular weight, concentration, ratio of interacting ionic materials, ionic strength (including modification of ionic strength, for example, by addition of salts), charge density of the cationic and anionic species, pH, and temperature. Coacervate systems and the effect of these parameters have been described, for example, by J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid and Interface Science, Vol. 140, No. 1, November 1990, pp 227-238, which descriptions are incorporated herein by reference.

Techniques for analyzing complex coacervates are known in the art. For example, microscopic analyses of the rinse-off hair coloring/cleansing compositions of the present invention, neat or at any chosen stage of dilution, can be utilized to identify whether a coacervate phase has formed. Such coacervate phase will be identifiable as an emulsified phase in the composition. The presence of dyes can aid in distinguishing the coacervate phase from other insoluble phases dispersed in the composition because the dye enhances the color difference thereby allowing the droplets to be easily seen.

The term “effective amount”, as used herein, with respect to coloring, refers to the amount necessary to impart the desired amount of color to hair under the usage conditions, and with respect to cleansing, refers to the amount necessary to clean hair under the usage conditions.

The term “substantially free of”, as used herein, refers to the level of the ingredient as being 0.01% or less of the composition.

### Components

The compositions of the present invention comprise as essential components suitable water-soluble dye materials, surfactant materials, and water as the carrier medium.

### The Water-Soluble Dye Materials

An essential component of the present invention is the water-soluble dye materials. In one embodiment, the cationic water-soluble dyes are preferably direct dyes. Direct dyes are commonly used in so-called “temporary” hair coloring products and in hair coloring products that are designed to gradually build-up or change the hair color. Direct dyes, in contrast to reactive dyes, do not undergo a chemical reaction in order to deliver color to the hair.

The water-soluble dye, oppositely charged from the surfactant, forms a coacervate with the surfactant. The direct water-soluble dyes that form a coacervate with the anionic and/or amphoteric surfactants are the cationic dyes. The amine functional dyes at a pH in which they are in substantially cationic form can also be used in the same manner. In another embodiment, the anionic and nonionic dyes do not form a coacervate; however, they preferentially partition into the separate amorphous liquid or liquid crystal phase formed by the surfactant materials. This also allows the dye to concentrate and thereby enhances its deposition.

The concentration of the water-soluble dye component typically ranges from about 0.1% to about 10%, preferably from about 0.2% to about 5%, by weight of the composition.

Nonlimiting examples of suitable cationic dyes are Arianor® Steel Blue (Basic Blue No. 99, CI number 56059); Arianor® Madder Red (Basic Red No. 76, CI number 12245); Arianor® Mahogany (Basic Brown No. 16, CI number 12550); Arianor® Straw Yellow (Basic Yellow No. 57, CI number 12719); Basic Yellow No. 11 (CI number 48055); Basic Red No. 29 (CI number 11460); Basic Red No. 12 (CI number 48070); Basic Blue No. 12 (CI number 51180); Basic Blue No. 3 (CI number 51104); Basic Violet No. 3 (CI number 42555); Basic Violet No. 16 (CI number 48013); Basic Green No. 4 (CI number 42000); and Arianor® Sienna Brown (Basic Brown No. 17, CI number 12251), and mixtures thereof, although this list is by no means comprehensive. (CI number is the number assigned to a dye in the Colour Index, 3^{rd} ed., Society of Dyers and Colourists, England, 1971, and fully identifies the chemical structures of the dye molecule.)

Nonlimiting examples of suitable anionic dyes are FD&C Blue No. 1 (CAS number 384445-9); FD&C Yellow No. 6 (CAS number 2783-94-0); FD&C Yellow No. 5 (CAS number 1934-21-0); FD&C Red 4 (CAS number 4548-53-2); FD&C Red No. 40 (CAS number 25956-17-6); D&C Brown No. 1 (CAS number 1320-7-6); and D&C Green No. 5 (CAS number 4403-90-1), and mixtures thereof. (CAS number is the number assigned to a dye in the Chemical Abstract Service Registry, and fully identifies the chemical structures of the dye molecule.)

Nonlimiting examples of suitable nonionic dyes are D&C Violet No. 2 (CAS number 81-48-1); D&C Yellow No. 7 (CAS number 2321-07-5); and D&C Red No. 36 (CAS number 2814-77-9), and mixtures thereof.

Nonlimiting examples of suitable amine functional dyes are HC Blue No. 2 (CAS number 33229-344); HC Blue No. 11 (CAS number 23920-15-2); HC Brown No. 1 (CAS number 83803-98-9); HC Green No. 1 (CAS number 52136-25-1); HC Red No. 3 (CAS number 2871-014); HC Red No. 8 (CAS number 13556-29-1); HC Violet No. 2 (CAS number n/a); and HC Yellow No. 4 (CAS number 5982043-8), and mixtures thereof.

Other suitable direct dyes may be found in the International Cosmetic Ingredient Dictionary and Handbook, Seventh Edition, 1997.

The preferred dyes for coacervate and/or liquid crystal formation with the surfactant component of the present invention are the cationic dyes listed above.

### The Surfactant Materials

The rinse-off hair coloring/cleansing compositions of the present invention comprise a surfactant materials component to provide cleaning performance to the composition and to participate in the formation of the dye-containing separated phases. The surfactant component comprises primarily one or more anionic surfactants, and/or one or more amphoteric surfactant. The surfactant component may additionally comprise one or more cationic surfactants, one or more nonionic surfactants or a combination thereof. Such surfactants should be physically and chemically compatible with the essential components described herein, and should not otherwise unduly impair product stability, aesthetics or performance.

The concentration of the surfactant materials component in the rinse-off hair coloring/cleansing composition should be sufficient to provide the desired cleaning and lather performance, and generally ranges from about 5% to about 50%, preferably from about 10% to about 25%, and most preferably from about 14% to about 20%, by weight of the composition.

Anionic Surfactant

Suitable anionic surfactant components for use in the rinse-off hair coloring/cleansing compositions herein include those which are known for use in hair care and other personal care cleansing compositions.

Suitable anionic surfactants include alkyl sulfates, alkyl ethoxylated sulfates, and mixtures thereof. These materials have the respective formulae (I) ROSO₃M and (II) RO(C₂H₄O)ₓSO₃M, wherein R is alkyl or alkenyl of from about 8 to about 30 carbon atoms, x is 1 to 10, and M is H or a salt-forming cation such as ammonium, alkanolamine containing C₁-C₃ alkyl groups such as triethanolamonium, and monovalent and polyvalent metals such as the alkali and alkaline earth metals. Preferred metals include sodium, potassium, magnesium, and calcium. The cation M, of the anionic surfactant should preferably be chosen such that the anionic surfactant component is water soluble. The solubility of anionic surfactants, in general, will depend upon the particular anionic surfactants and cations chosen. As an aid to determining appropriate mixtures of anionic surfactants, the anionic surfactants should be chosen such that the Krafft temperature is about 15° C. or less, preferably about 10° C. or less, more preferably about 0° C. or less. It is also preferred that the anionic surfactant not crystallize out of the composition under storage conditions.

In formulas (I) and (II), R preferably has from about 10 to about 18 carbon atoms in both the alkyl and alkyl ethoxylated sulfates. The alkyl ethoxylated sulfates are typically made as condensation products of ethylene oxide and monohydric alcohols having from about 8 to about 30 carbon atoms. The alcohols can be derived from fats, e.g., coconut oil, palm kernel oil, or tallow, or can be synthetic. Such alcohols are preferably reacted with about 1 to about 10, more preferably from about 1 to about 4, most preferably from about 2 to about 3.5, molar proportions of ethylene oxide. The resulting mixture of molecular species having, for example, an average of 3 moles of ethylene oxide per mole of alcohol, is sulfated and neutralized.

Specific examples of alkyl ether sulfates which may be used in the present invention are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 4 moles of ethylene oxide.

Specific suitable anionic surfactants include, but are not limited to water-soluble salts of organic, sulfuric acid reaction products of the general formula (I) where R₁ is selected from the group consisting of straight or branched chain, saturated aliphatic hydrocarbon radicals having from about 8 to about 24, preferably about 10 to about 18, carbon atoms, and M is a cation such as ammonium, alkanolamines, such as triethanolamine, monovalent metal cations, such as sodium and potassium, and polyvalent metal cations, such as magnesium, and calcium. The cation M, of the anionic detersive surfactant should be chosen such that the surfactant component is water soluble. Solubility will depend upon the particular anionic surfactants and cations chosen. Examples of such surfactants are the salts of an organic sulfuric acid reaction product of a hydrocarbon of the methane series, including iso-, neo-, and n-paraffins, having about 8 to about 24 carbon atoms, preferably about 10 to about 18 carbon atoms and a sulfonating agent, e.g., SO₃, H₂SO₄, obtained according to known sulfonation methods, including bleaching and hydrolysis. Preferred are alkali metal and ammonium sulfonated C₁₀₋₁₈ n-paraffins.

The sulfate surfactant preferably comprises a combination of ethoxylated and non-ethoxylated sulfates. There is no required ratio of ethoxylated to non-ethoxylated sulfates; however, a ratio of greater than 1:1 is preferred. Alkyl sulfates provide excellent cleaning and lather performance. Alkyl ethoxylated sulfates provide excellent cleaning performance and are mild to the skin and to the eyes.

Another class of anionic surfactants suitable for use in the present invention are the reaction products of fatty acids esterified with isethionic acid and neutralized with sodium hydroxide where, for example, the fatty acids are derived from coconut oil or palm kernel oil; sodium, ammonium, tetraethylammonium or potassium salts of fatty acid amides of methyl tauride in which the fatty acids, for example, are derived from coconut oil or palm kernel oil. Other similar anionic surfactants are described in U.S. Pat. No. 2,486,921; U.S. Pat. No. 2,486,922; and U.S. Pat. No. 2,396,278, which descriptions are incorporated herein by reference.

Specific anionic surfactants suitable for use in the present invention are the succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; dioctyl esters of sodium sulfosuccinic acid.

Other suitable anionic surfactants include alkyl and alkenyl glyceryl ether sulfonate surfactants (also referred to herein as an “AGS” surfactant), derivatives thereof and salts thereof. These AGS surfactants are derived from an alkyl glyceryl ether containing a sulfonate or sulfonate salt group. These compounds generally can be described as an alkyl monoether of glycerol that also contains a sulfonate group.

These AGS surfactants can be described as generally conforming to the following structures:

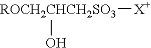

and

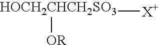

wherein R is a saturated or unsaturated straight chain, branched chain, or cyclic hydrocarbyl group having from about 10 to about 18 carbon atoms, preferably from about 11 to about 16 carbon atoms, and most preferably from about 12 to about 14 carbon atoms, and X is a cation selected from the group consisting of ammonium; mono-alkylsubstituted ammonium; di-alkylsubstituted ammonium; tri-alkylsubstituted ammonium; tetra-alkylsubstituted ammonium; alkali metal; alkaline earth metal; and mixtures thereof. More preferably, the alkyl radicals, R in the above formulas, are saturated and straight chain.

Still other suitable anionic surfactants include olefin sulfonates having about 10 to about 24 carbon atoms. The term “olefin sulfonates” is used herein to mean compounds which can be produced by the sulfonation of alpha-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A specific alpha-olefin sulfonate mixture of the above type is described more fully in the U.S. Pat. No. 3,332,880, which description is incorporated herein by reference.

The alpha-olefins from which the olefin sulfonates are derived are mono-olefins having about 10 to about 24 carbon atoms, preferably about 12 to about 16 carbon atoms. Preferably, they are straight chain olefins.

Another class of anionic surfactants suitable for use in the present invention are the beta-alkyloxy alkane sulfonates. These compounds have the following formula:

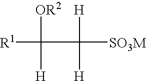

where R¹ is a straight chain alkyl group having from about 6 to about 20 carbon atoms, R² is a lower alkyl group preferably having from about 1 to about 3 carbon atoms, and M is a water-soluble cation as hereinbefore described.

Specific examples of highly preferred anionic surfactants suitable for use in the present invention include alkyl glyceryl ether sulfonate, ammonium lauryl sulfate, ammonium laureth sulfate, triethylamine lauryl sulfate, triethylamine laureth sulfate, triethanolamine laureth sulfate, monoethanolamine laureth sulfate, diethanolamine lauryl sulfate, diethanolamine laureth sulfate, lauric monoglyceride sodium sulfate, sodium laureth sulfate, potassium laureth sulfate, sodium lauryl sarcosinate, sodium lauryl sarcosinate, lauryl sarcosine, cocoyl sarcosine, ammonium cocoyl sulfate, ammonium lauryl sulfate, sodium cocoyl sulfate, sodium lauryl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and mixtures thereof.

The concentration of the anionic surfactant portion in the surfactant materials component of the composition generally ranges from about 0% to about 100%, preferably from about 20% to about 100%, by weight of the surfactant materials component.

Amphoteric Surfactant

Suitable amphoteric surfactants for use in the rinse-off hair coloring/cleansing compositions herein include those which are known for use in hair care or other personal care cleansing compositions.

Examples of amphoteric surfactants which can be used in the compositions of the present invention are sodium 3-dodecyl-aminopropionate, sodium 3-dodecylaminopropane sulfonate, sodium lauroamphoacetate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name “MIRANOL”™ and described in U.S. Pat. No. 2,528,378.

Other amphoterics (sometimes classified as zwitterionics) such as betaines can also be used in the present invention. Such zwitterionics are considered as amphoterics in the present invention where the zwitterionic has an attached group that is anionic at the pH of the composition. Examples of betaines useful herein include the high alkyl betaines, such as coco dimethyl carboxymethyl betaine, cocoamidopropyl betaine, cocobetaine, lauryl arnidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. Useful sulfobetaines include coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃-radical is attached to the nitrogen atom of the betaine are also useful in this invention. Most preferred for use herein is cocoamidopropyl betaine and sodium lauroamphoacetate.

The concentration of the amphoteric surfactant portion of the surfactant materials generally range from about 0% to about 100%, preferably from about 20% to about 80%, by weight of the surfactant materials component.

Cationic and Nonionic Surfactants

The surfactant materials of the present invention may optionally include nonionic surfactants, cationic surfactants, and combinations thereof. Suitable classes of nonionic surfactants include:

1. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of about 2,500 to about 3,000, are satisfactory.

2. The condensation product of aliphatic alcohols having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms.

3. Long chain tertiary amine oxides are classified for the present purposes as nonionic surfactants, although they are typically referred to as “semi-polar”. Such surfactants correspond to the following general formula:

R₁R₂R₃N→0

wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semi-polar bond. Examples of amine oxides suitable for use in this invention include dimethyldodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyl-dimethylamine oxide, 3-dodecoxy-2-hydroxypropyldi(3-hydroxy-propyl) amine oxide, dimethylhexadecylamine oxide.

4. Long chain tertiary phosphine oxides corresponding to the following general formula:

RR′R″P→0

wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms. The arrow in the formula is a conventional representation of a semi-polar bond. Examples of suitable phosphine oxides are: dodecyl-dimethylphosphine oxide, tetradecyldimethylphosphine oxide, tetradecylmethylethylphosphine oxide, 3,6,9,-trioxaoctadecyl-dimethylphosphine oxide, cetyldimethylphosphine oxide, 3-dodecoxy-2-hydroxypropyldi(2-hydroxyethyl) phosphine oxide, stearyldimethylphosphine oxide, cetylethylpropylphosphine oxide, oleyldiethylphosphine oxide, dodecyldiethylphosphine oxide, tetradecyldiethylphosphine oxide, dodecyl-dipropylphosphine oxide, dodecyldi(hydroxymethyl)phosphine oxide, dodecyldi(2-hydroxy-ethyl)phosphine oxide, tetradecylmethyl-2-hydroxy-propylphosphine oxide, oleydimethylphosphine oxide, 2-hydroxydodecyl-dimethyl-phosphine oxide.

5. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety. Examples include: octadecyl methyl sulfoxide, 2-ketotridecyl methyl sulfoxide, 3,6,9,-trixaoctadecyl 2-hydroxyethyl sulfoxide, dodecyl methyl sulfoxide, oleyl 3-hydroxypropyl sulfoxide, tetradecyl methyl sulfoxide, 3-methoxytridecyl methyl sulfoxide, 3-hydroxytridecyl methyl sulfoxide, 3-hydroxy-4-dodecoxybutyl methyl sulfoxide.

6. Polyalkylene oxide modified dimethylpolysiloxanes, also known as dimethicone copolyols. These materials include the polyalkylene oxide modified dimethylpolysiloxanes of the following formulae:

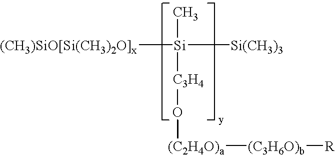

and

R′—Si—[[O—Si—(CH₃)₂]ₓ(OC₂H₄)ₐ—(OC₃H₆)_{b}—OR″]₃

wherein R is hydrogen, an alkyl group having from 1 to about 12 carbon atoms, or an alkoxy group having from 1 to about 6 carbon atoms; R′ and R″ are alkyl groups having from 1 to about 12 carbon atoms; x is an integer of from 1 to 100, preferably from 20 to 30; y is an integer of 1 to 20, preferably from 2 to 10; and a and b are integers of from 1 to 50, preferably from 20 to 30. Dimethicone copolyols among those useful herein are disclosed in the following patent documents, all incorporated by reference herein: U.S. Pat. No. 4,122,029, Gee et al., issued Oct. 24, 1978; U.S. Pat. No. 4,265,878, Keil, issued May 5, 1981; and U.S. Pat. No. 4,421,769, Dixon et al., issued Dec. 20, 1983. Commercially available dimethicone copolyols, useful herein, include Silwet Surface Active Copolymers (manufactured by the Union Carbide Corporation); Dow Corning Silicone Surfactants (manufactured by the Dow Corning Corporation); Silicone Copolymer F-754 (manufactured by SWS Silicones Corp.); and Rhodorsil 70646 Fluid (manufactured by Rhone Poulenc, Inc.).

The rinse-off hair coloring/cleansing compositions of the present invention can optionally comprise select cationic materials for use as surfactants. The surfactants for use in the composition are select quaternary ammonium or protonated amino compounds. The select surfactants are cationic and must be positively charged at the pH of the shampoo compositions. Generally, the pH of the shampoo composition will be less than about 10, typically from about 3 to about 9, preferably from about 4 to about 8, measured neat.

Select cationic surfactants for use in the composition include those corresponding to the to the formula:

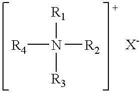

wherein R₁, and R₂ are independently a saturated or unsaturated, substituted or unsubstituted, linear or branched hydrocarbon chain having from about 12 to about 30 carbon atoms, preferably from about 18 to about 22 carbon atoms, and wherein the hydrocarbon chain can contain one or more hydrophilic moieties selected from the alkoxy, polyoxyalkylene, alkylamido, hydroxyalkyl, alkylester, and mixtures thereof; R₃ and R₄ are independently a hydrogen, or a saturated or unsaturated, substituted or unsubstituted, linear or branched hydrocarbon chain having from about 1 to about 30 carbon atoms, or a hydrocarbon having from about 1 to about 30 carbon atoms containing one or more aromatic, ester, ether, amido, amino moieties present as substituents or as linkages in the chain, and wherein the hydrocarbon chain can contain one or more hydrophilic moieties selected from the alkoxy, polyoxyalkylene, alkylamido, hydroxyalkyl, alkylester, and mixtures thereof; and X is a soluble salt forming anion preferably selected from halogen (especially chlorine), acetate, phosphate, nitrate, sulfonate, and alkylsulfate radicals.

Nonlimiting examples of suitable cationic surfactants include ditallowamidoethyl hydroxypropylmonium methosulfate (commercially available as Varisoft® 238), dihydrogenated tallowamidoethyl hydroxyethylmonium methosulfate (commercially available as Varisoft® 110), ditallowamidoethyl hydroxyethylmonium methosulfate (commercially available as Varisoft® 222), and di(partially hardened soyoylethyl) hydroxyethylmonium methosulfate (commercially available as Armocare® EQ-S), detyltrimethylammonium chloride, ditallowamidoethyl hydroxypropylmonium methosulfate, dihydrogenated tallowamidoethyl hydroxyethylmonium methosulfate, ditallowamidoethyl hydroxyethylmonium methosulfate, and di(partially hardened soyoylethyl) hydroxyethylmonium methosulfate, methyl bis(2-hydroxyethyl) coco-ammonium chloride, methyl bis(2-hydroxyethyl) tallow ammonium chloride, methyl bis(2-hydroxyethyl) oleyl ammonium chloride, cocomethyl bis(2-hydroxyethyl) ammonium chloride, methyl bis(oleyl-amidoethyl) 2-hydroxyethyl ammonium methyl sulfate, dilauryl acetyl dimonium chloride, hydroxycetyl hydroxyethyl dimonium chloride, cetyl trimonium chloride, isostearamidopropyl laurylacetodimonium chloride, and Quaternium 26®—represented by Formula I, where RCO represents fatty acid groups derived from mink oil, and mixtures thereof,

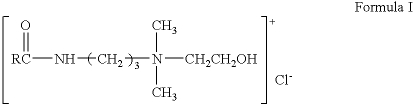

are particularly preferred quaternary ammonium cationic surfactants useful herein.

Although useful in the compositions of the present invention, nonionic or cationic surfactants tend to reduce the lathering properties of shampoo compositions. To maintain adequate lathering profiles, nonionic or cationic surfactants, if present, are used at low concentrations. If used, the surfactant materials of the present invention will contain less than about 3%, more preferably less than about 1%, of the nonionic and cationic surfactants.

The weight ratio of dye materials to surfactant materials is generally in the range from about 1:500 to about 2:1, preferably from about 1:12.5 to about 1:2.

### Non-Surfactant Electrolytes

Non-surfactant electrolytes may be necessary in certain embodiments of the present invention to aid in thickening the product and to help initiate separation of a phase into which the dyes preferentially partition.

Suitable non-surfactant electrolytes include mono-, di- and trivalent inorganic salts as well as organic salts. Surfactant salts, themselves, are not included in the present electrolyte definition, but other salts are. Suitable salts include, but are not limited to, phosphates, sulfates, nitrates, citrates and halides. The counterions of such salts can be, but are not limited to, sodium, potassium, ammonium, magnesium or other mono-, di-and tri-valent cations. Non-surfactant electrolytes most preferred for use in the compositions of the present invention include sodium chloride, ammonium chloride, sodium citrate, and magnesium sulfate. The amount of the electrolyte used will generally depend on the amount of the amphiphile incorporated, but may be used at concentration levels of from about 0.1% to about 10%, preferably from about 0.2% to about 4%.

The surfactants and dyes of this invention must be chosen so as to form a separated liquid phase in which the dye is concentrated. The separated phase needs to be liquid so that it will deposit readily and spread readily on the hair. This separated phase may be formed as a complex 8048C between the surfactant materials and the dye materials. The separated phase may also be formed by the surfactant materials themselves, or their interaction with electrolytes or amphiphiles in the shampoo. The separated phase should have a droplet size from about 0.5 μm to about 100 μm in diameter. Although droplets larger than 100 μm will give enhanced coloring, they may give uneven appearance to the hair, or be unstable, causing separation into layers of the finished product. The concentration of the dye into the separated phase is visually distinct, and can be confirmed by microscopic observation of the greater color intensity in the droplets than in the continuous phase.

### Water

The rinse-off hair coloring/cleansing compositions of the present invention are aqueous systems which comprise from about 40% to about 94.9%, preferably from about 50% to about 90%, more preferably from about 60% to about 85%, water by weight of the composition, as the carrier.

### Preferred Optional Components

### Silicones

The compositions of the present invention may optionally include non-surfactant silicone conditioning components. Typically, the silicone components are intermixed into coloring/cleansing compositions, forming a separate, discontinuous silicone phase. The silicone conditioning component will comprise a silicone fluid conditioning agent such as a silicone fluid and can also comprise other ingredients, such as a silicone gum or resin to enhance silicone fluid deposition efficiency or enhance glossiness of the hair, especially when high refractive index (e.g. above about 1.46) silicone conditioning agents are used (e.g., highly phenylated silicones).

The silicone conditioning agent phase may comprise volatile silicones, nonvolatile silicones, or mixtures thereof. As used herein, “nonvolatile” refers to silicone material with little or no significant vapor pressure under ambient conditions, as is understood by those in the art. Boiling point under one atmosphere will preferably be at least about 250° C., more preferably at least about 275° C., most preferably at least about 300° C. Vapor pressure is preferably about 0.2 mm Hg at 25° C. or less, preferably about 0.1 mm Hg at 25° C. or less. Typically, if volatile silicones are present, it will be incidental to their use as a solvent or carrier for commercially available forms of nonvolatile silicone materials ingredients, such as silicone gums and resins.

The silicone conditioning agents for use in the compositions of the present invention preferably have a viscosity of from about 20 to about 2,000,000 centistokes, more preferably from about 1,000 to about 1,800,000 centistokes, even more preferably from about 10,000 to about 1,500,000 centistokes, most preferably from about 30,000 to about 1,000,000 centistokes, at 25° C. The viscosity can be measured by means of a glass capillary viscometer as set forth in Dow Coming Corporate Test Method CTM0004, Jul. 20, 1970.

Optional silicone fluids for use in the present compositions include silicone oils which are flowable silicone materials with a viscosity of less than 1,000,000 centistokes, preferably between about 5 and 1,000,000 centistokes, more preferably between about 10 and about 600,000 centistokes, more preferably between about 10 and about 500,000 centistokes, most preferably between 10 and 300,000 centistokes at 25° C. Suitable silicone oils include polyalkyl siloxanes, polyaryl siloxanes, polyalkylaryl siloxanes, polyether siloxane copolymers, and mixtures thereof. Other insoluble, nonvolatile silicone fluids having conditioning properties can also be used.

Optional silicone oils for use in the composition include polyalkyl or polyaryl siloxanes which conform to following formula:

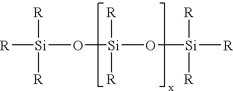

where R is aliphatic, preferably alkyl or alkenyl, or aryl, R can be substituted or unsubstituted, and x is an integer from 1 to about 8,000. Suitable unsubstituted R groups include alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

The aliphatic or aryl groups substituted on the siloxane chain may have any structure as long as the resulting silicones remain fluid at room temperature, are hydrophobic, are neither irritating, toxic nor otherwise harmful when applied to the hair or skin, are compatible with the other components of the herein described coloring/cleansing compositions, are chemically stable under normal use and storage conditions, are insoluble in the compositions of the present invention, and are capable of being deposited on and, of conditioning, the hair.

The two R groups on the silicon atom of each monomeric silicone unit may represent the same group or different groups. Preferably, the two R groups represent the same group.

Preferred alkyl and alkenyl substituents are C₁-C₅ alkyls and alkenyls, more preferably from C₁-C₄, most preferably from C₁-C₂. The aliphatic portions of other alkyl-, alkenyl-, or alkynyl-containing groups (such as alkoxy, alkaryl, and alkamino) can be straight or branched chains and preferably have from one to five carbon atoms, more preferably from one to four carbon atoms, even more preferably from one to three carbon atoms, most preferably from one to two carbon atoms. As discussed above, the R substituents hereof can also contain amino functionalities, e.g. alkamino groups, which can be primary, secondary or tertiary amines or quaternary ammonium. These include mono-, di- and tri-alkylamino and alkoxyamino groups wherein the aliphatic portion chain length is preferably as described above. The R substituents can also be substituted with other groups, such as halogens (e.g. chloride, fluoride, and bromide), halogenated aliphatic or aryl groups, and hydroxy (e.g. hydroxy substituted aliphatic groups). Suitable halogenated R groups could include, for example, tri-halogenated (preferably fluoro) alkyl groups such as —R¹—C(F)₃, wherein R¹ is C₁-C₃ alkyl. Examples of such polysiloxanes include polymethyl-3,3,3 trifluoropropylsiloxane.

Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred. Other suitable R groups include methyl, methoxy, ethoxy, propoxy, and aryloxy. The three R groups on the end caps of the silicone may also represent the same or different groups.

The nonvolatile polyalkylsiloxane fluids that may be used include, for example, polydimethylsiloxanes. These siloxanes are available, for example, from the General Electric Company in their Viscasil R and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level must be sufficiently low to prevent solubility in water and the composition hereof.

Other suitable silicone fluids for use in the silicone conditioning agents are insoluble silicone gums. These gums are polyorganosiloxane materials having a viscosity at 25° C. of greater than or equal to 1,000,000 centistokes. Silicone gums are described in U.S. Pat. No. 4,152,416; Noll and Walter, Chemistry and Technology of Silicones, New York: Academic Press 1968; and in General Electric Silicone Rubber Product Data Sheets SE 30, SE 33, SE 54 and SE 76, all of which are incorporated herein by reference. The silicone gums will typically have a mass molecular weight in excess of about 200,000, generally between about 200,000 and about 1,000,000, specific examples of which include polydimethylsiloxane, (polydimethylsiloxane) (methylvinylsiloxane) copolymer, poly(dimethylsiloxane) (diphenyl siloxane)(methylvinylsiloxane) copolymer, and mixtures thereof.

The silicone conditioning agent can also comprise a mixture of polydimethylsiloxane gum (viscosity greater than about 1,000,000 centistokes) and polydimethylsiloxane oil (viscosity from about 10 to about 100,000 centistokes), wherein the ratio of gum to fluid is from about 30:70 to about 70:30, preferably from about 40:60 to about 60:40.

References disclosing examples of some suitable silicone fluids for use in the personal cleansing compositions include U.S. Pat. No. 2,826,551, U.S. Pat. No. 3,964,500, U.S. Pat. No. 4,364,837, British Patent 849,433, and Silicon Compounds, Petrarch Systems, Inc. (1984), all of which are incorporated herein by reference.

Silicone resins can be included in the silicone conditioning agent. These resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Background material on silicones including sections discussing silicone fluids, gums, and resins, as well as manufacture of silicones, can be found in Encyclopedia of Polymer Science and Engineering, Volume 15, Second Edition, pp. 204-308, John Wiley & Sons, Inc., 1989, incorporated herein by reference.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MDT and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

The weight ratio of the nonvolatile silicone fluid, having refractive index below 1.46, to the silicone resin component, when used, is preferably from about 4:1 to about 400:1, preferably this ratio is from about 9:1 to about 200:1, more preferably from about 19:1 to about 100:1, particularly when the silicone fluid component is a polydimethylsiloxane fluid or a mixture of polydimethylsiloxane fluid and polydimethylsiloxane gum as described above. Insofar as the silicone resin forms a part of the same phase in the compositions hereof as the silicone fluid, i.e. the conditioning active, the sum of the fluid and resin should be included in determining the level of silicone conditioning agent in the composition.

The number average particle size of the optional silicone component can vary widely depending on the formulation and/or the desired characteristics. Number average particle sizes preferred for use in the present invention range from about 10 nanometers to about 100 microns, more preferably from about 30 nanometers to about 20 microns.

### Deposition Aid

The rinse-off hair coloring/cleansing compositions of the present invention may include an organic cationic polymer as a deposition aid for the droplets containing the concentrated dye. The organic cationic polymer contains cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the coloring/cleansing composition. The average molecular weight of the cationic polymer is between about 10 million and about 5,000, preferably at least about 100,000, more preferably at least about 200,000, but preferably not more than about 2 million, more preferably not more than about 1.5 million. The polymers also have a cationic charge density ranging from about 0.2 meq/gm to about 7 meq/gm, preferably at least about 0.4 meq/gm, more preferably at least about 0.6 meq/gm, but also preferably less than about 5 meq/gm, more preferably less than about 2 meq/gm. The charge density can be measured using the Kjeldahl method and should be within the above limits at the desired pH of use, which pH will generally range from about pH 3 to about pH 9, preferably from about pH 4 about pH 7.

Any anionic counterions can be used in association with the cationic polymers so long as the polymers remain soluble in water, in the rinse-off hair coloring/cleansing composition, or in a coacervate phase of the rinse-off hair coloring/cleansing composition, and so long as the counterions are physically and chemically compatible with the essential components of the rinse-off hair coloring/cleansing composition or do not otherwise unduly impair product performance, stability or aesthetics. Non limiting examples of such counterions include halides (e.g., chlorine, fluorine, bromine, iodine), sulfate and methylsulfate.

The cationic nitrogen-containing moiety of the cationic polymer is generally present as a substituent on all, or more typically on some, of the monomer units thereof. Thus, the cationic polymer for use in the rinse-off hair coloring/cleansing composition includes homopolymers, copolymers, terpolymers, and so forth, of quaternary ammonium or cationic amine-substituted monomer units, optionally in combination with non-cationic monomers referred to herein as spacer monomers. Non limiting examples of such polymers are described in the International Cosmetic Ingredient Dictionary and Handbook, Seventh Edition, (The Cosmetic, Toiletry, and Fragrance Association, Inc., Washington, D.C. (1997)), which description is incorporated herein by reference.

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. The alkyl and dialkyl substituted monomers preferably have from C₁ to C₇ alkyl groups, more preferably from C₁ to C₃ alkyl groups. Other suitable spacer monomers include vinyl esters, vinyl alcohol (made by hydrolysis of polyvinyl acetate), maleic anhydride, propylene glycol, and ethylene glycol.

Suitable cationic protonated amino and quaternary ammonium monomers, for inclusion in the cationic polymers of the rinse-off hair coloring/cleansing compositions herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁, C₂ or C₃ alkyls.

Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

Other suitable cationic polymers for use in the rinse-off hair coloring/cleansing composition include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., U.S.A.) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from ISP Corporation (Wayne, N.J., U.S.A.) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homopolymers and copolymers of unsaturated carboxylic acids having from 3 to 5 carbon atoms, as described in U.S. Pat. No. 4,009,256, which description is incorporated herein by reference.

Other suitable cationic polymers for use in the rinse-off hair coloring/cleansing composition include polysaccharide polymers, such as cationic cellulose derivatives and cationic starch derivatives. Suitable cationic polysaccharide polymers include those which conform to the formula

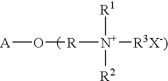

wherein A is an anhydroglucose residual group, such as a starch or cellulose anhydroglucose residual; R is an alkylene oxyalkylene, polyoxyalkylene, or hydroxyalkylene group, or combination thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are those polymers available from Amerchol Corp. (Edison, N.J., USA) in their Polymer JR and LR series of polymers, as salts of hydroxyethyl cellulose reacted with trimethyl ammonium substituted epoxide, referred to in the industry (CTFA) as Polyquaternium 10. Another type of preferred cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the trade name Polymer LM-200.

Other suitable cationic polymers include cationic guar gum derivatives, such as guar hydroxypropyltrimonium chloride, specific examples of which include the Jaguar series commercially available from Rhone-Poulenc Incorporated. Other suitable cationic polymers include quaternary nitrogen-containing cellulose ethers, some examples of which are described in U.S. Pat. No. 3,962,418, which description is incorporated herein by reference herein. Other suitable cationic polymers include copolymers of etherified cellulose, guar and starch, some examples of which are described in U.S. Pat. No. 3,958,581, which description is incorporated herein by reference.

The cationic polymers herein are either soluble in the rinse-off hair coloring/cleansing composition, or preferably are soluble in a complex coacervate phase in the rinse-off hair coloring/cleansing composition formed by the cationic polymer and the anionic surfactant component described hereinbefore. Complex coacervates of the cationic polymer can also be formed with other charged materials in the rinse-off hair coloring/cleansing composition.

Complex coacervates are believed to more readily deposit on the hair, and to trap other dispersed materials and aid their depositions. Thus, in general, it is preferred that the cationic polymer exist in the rinse-off hair coloring composition as a coacervate phase or form a coacervate phase upon dilution.

If used, the concentration of the cationic polymer component in the rinse-off hair coloring/cleansing composition generally ranges from about 0.02% to about 10%, preferably from about 0. 1% to about 2%, by weight of the composition.

### Suspending Agents

The rinse-off hair coloring/cleansing compositions of the present invention may optionally comprise a suspending agent at concentrations effective for suspending the separated dye phase and the optional silicone hair conditioning agent, or other water-insoluble material, in dispersed form in the cleansing compositions. Usage levels for the suspending agents range from about 0.1% to about 10%, preferably from about 0.3% to about 5.0%, by weight of the cleansing compositions.

Optional suspending agents include crystalline suspending agents that can be categorized as acyl derivatives, long chain amine oxides, or combinations thereof, concentrations of which range from about 0.1% to about 5.0%, preferably from about 0.5% to about 3.0%, by weight of the cleansing compositions. When used in the cleansing compositions, these suspending agents are present in crystalline form. These suspending agents are described in U.S. Pat. No. RE 34,584, which description is incorporated herein by reference. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); glyceryl esters (e.g., glyceryl distearate) and long chain esters of long chain alkanol amides (e.g., stearamide diethanolamide distearate, stearamide monoethanolamide stearate). Long chain acyl derivatives, ethylene glycol esters of long chain carboxylic acids, long chain amine oxides, and alkanol amides of long chain carboxylic acids in addition to the preferred materials listed above may be used as suspending agents. For example, it is contemplated that suspending agents with long chain hydrocarbyls having C₈-C₂₂ chains may be used.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan Company (Northfield, Ill., USA).

Examples of suitable long chain amine oxides for use as suspending agents include alkyl (C₁₆-C₂₂) dimethyl amine oxides, e.g., stearyl dimethyl amine oxide.

Other suitable suspending agents include xanthan gum at concentrations ranging from about 0.3% to about 3%, preferably from about 0.4% to about 1.2%, by weight of the cleansing compositions. The use of xanthan gum as a suspending agent in silicone containing cleansing compositions is described, for example, in U.S. Pat. No. 4,788,006, which description is incorporated herein by reference. Combinations of long chain acyl derivatives and xanthan gum may also be used as a suspending agent in the cleansing compositions. Such combinations are described in U.S. Pat. No. 4,704,272, which description is incorporated herein by reference.

Other suitable suspending agents include carboxyvinyl polymers. Preferred among these polymers are the copolymers of acrylic acid crosslinked with polyallylsucrose as described in U.S. Pat. No. 2,798,053, which description is incorporated herein by reference. Examples of these polymers include Carbopol 934, 940, 941, and 956, available from B. F. Goodrich Company.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

Other suitable suspending agents may be used in the cleansing compositions, including those that can impart a gel-like viscosity to the composition, such as water soluble or colloidally water soluble polymers like cellulose ethers (e.g., methylcellulose, hydroxybutyl methylcellulose, hyroxypropylcellulose, hydroxypropyl methylcellulose, hydroxyethyl ethylcellulose and hydorxethylcellulose), guar gum, polyvinyl alcohol, polyvinyl pyrrolidone, hydroxypropyl guar gum, starch and starch derivatives, other thickeners, viscosity modifiers, gelling agents, etc. Mixtures of these materials can also be used.

### Other Optional Components

In addition to the components described hereinbefore, the rinse-off coloring/cleansing compositions of the present invention may further comprise one or more optional components known for use in hair care or personal care products, provided that the optional components are physically and chemically compatible with the essential components described herein, or do not otherwise unduly impair product stability, aesthetics or performance.

Nonlimiting examples of such optional components are disclosed in International Cosmetic Ingredient Dictionary and Handbook, Seventh Edition, 1997, which is incorporated by reference herein in its entirety. Some nonlimiting examples of such optional components are fatty alcohols; styling agents; perfumes; preservatives (e.g., benzoic acid, methyl paraben, propyl paraben, imidazolidinyl urea, iodopropynyl butyl carbamate, methylisothiazolinone, methylchloroisothiazolinone); lather boosters (e.g., polyalkylene glycol and cocomonoethanolamide); anti-static agents; anti-dandruff agents (e.g., pyridinethione salts and selenium compounds); viscosity adjusting agents; thickeners; pH adjusting agents (e.g., sodium citrate, citric acid, succinic acid, phosphoric acid, sodium hydroxide, and sodium carbonate); anti-microbial agents (e.g., Triclosan® or triclocarbon); anti-oxidants; diluents; pearlescent aids; proteins; scalp sensates (e.g., menthol); topical anesthetics; skin active agents; sunscreening agents; vitamins; humectants; and pediculocides (e.g., pyrethrins).

### Method of Manufacture

The compositions of the present invention, in general, can be prepared by combining, i.e., by mixing together at elevated temperature, e.g., about 72° C., water and a portion of the surfactants along with any solids (e.g., amphiphiles) that need to be melted, to speed mixing of the solids into the water and surfactant composition. The dye materials and additional ingredients including any electrolytes can be added either to this hot premix or after cooling the premix. The cationic polymers can be added as a water solution after cooling the premix. The ingredients are mixed thoroughly at the elevated temperature and then optionally pumped through a high shear mill and then through a heat exchanger to cool them to ambient temperature. If used, the silicone conditioner may be emulsified at room temperature in undiluted surfactant and then added to the cooled product. Alternately, the silicone conditioning agent can be mixed with the anionic surfactant and fatty alcohol, such as cetyl and stearyl alcohols, at elevated temperature, to form a premix containing dispersed silicone. The premix can then be added to and mixed with the remaining materials of the composition, pumped through a high shear mill, and cooled.

### Method of Use

The rinse-off coloring/cleansing compositions of the present invention are used in a conventional manner to color and cleanse hair.

This method for coloring and cleansing the hair comprises the steps of: a) wetting the hair with water, b) applying an effective amount of the coloring composition to the hair, where -the effective amount generally ranges from about 1 g. to about 50 g., preferably from about 1 g. to about 20 g., c) working the composition throughout the hair, and d) rinsing the coloring composition from the hair using water. Working the composition throughout the hair is in accordance to the manner used in a standard shampooing operation. The composition is allowed to stand in contact with the hair for about 3 seconds to about 5 minutes before rinsing; however, a longer time period may be used. These steps can be repeated as many times as desired to achieve the coloring and cleansing benefits. The repetition of this process yields a more intense coloration of the hair.

### EXAMPLES

The coloring shampoos of Examples I-III illustrate specific embodiments of the phase-separated rinse-off hair coloring/cleansing compositions of the present invention, but are not intended to be limiting thereof. Other modifications can be undertaken by the skilled artisan without departing from the spirit and scope of this invention. Ingredients are hereinafter identified by chemical, trade or The Cosmetic, Toiletry, and Fragrance Association (CTFA) name.

Examples I and II can be made by simply mixing the ingredients together. Example III can be made in the following manner: 1) mixing together the water, the ammonium laureth sulfate, the ammonium lauryl sulfate, the ethylene glycol distearate and the cocamide DEA at 73° C.; 2) pumping this mixture through a high shear mixer and then through a heat exchanger to cool; 3) adding the dimethicone which is prepared with some of the surfactant (70:30 dimethicone to ammonium laureth sulfate (25% active)) as a premix; 4) mixing in the perfume and the DMDM Hydantoin; 5) making a premix of Polycare 133 at 3.3% in water, and adding it slowly to the other ingredients.

### EXAMPLE I

| Ingredient | Amount (weight percent) |
|---|---|
| Sodium lauroamphoacetate | 16 |
| Basic Blue No. 99 | 3 |
| Water | q.s. |

### EXAMPLE II

| Ingredient | Amount (weight percent) |
|---|---|
| Ammonium Lauryl Sulfate | 16 |
| Basic Brown No. 17 | 3 |
| NaCl | 4 |
| Water | q.s. |

### EXAMPLE III

| Ingredient | Amount (weight percent) |
|---|---|
| Ammonium Laureth Sulfate¹ | 6.5 |
| Ammonium Lauryl Sulfate | 5.5 |
| Basic Blue No. 99 | 3.0 |
| Ethylene Glycol Distearate | 1.5 |
| Cocamide MEA | 0.8 |
| Dimethicone | 1.0 |
| Perfume | 0.4 |
| DMDM Hydantoin | 0.37 |
| Polycare 133² | 0.40 |
| Water | q.s. |

¹Ammonium Laureth Sulfate is CH₃(CH₂)₁₀CH₂(OCH₂CH₂)ₙOSO₃NH₄, where n is between 1 and 4.
²Polycare 133 is Polymethacrylamidopropyl Trimonium Chloride, from Rhone-Poulenc.

The rinse-off hair coloring/cleansing compositions illustrated in Examples I-III provide excellent rinse-off, even coloring, cleansing and mildness, and do not stain or irritate the skin.

The composition of Example I, examined microscopically (at 620×), shows individual dark blue (nearly black), spherical droplets ranging from about 3 μm to about 60 μm in diameter and a continuous background phase that is very light blue.

The composition of Example II, examined microscopically (at 620×), shows aggregates of yellowish brown droplets which individually range from about 10 μm to about 100 μm in diameter. Individual droplets of about 6 μm to about 30 μm in diameter, which have only a little of the brown color, are also visible. The continuous phase has no discernible brown color.

The composition of Example III, examined microscopically (at 620×), shows several different phases. There are medium to dark blue droplets of about 10 μm to about 50 μm in diameter aggregated around a large (about 150 μm in diameter) colorless silicone drop; droplets are also seen aggregated in other areas. Crystals of the ethylene glycol distearate suspending agents are also visible. The background continuous phase is light blue.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A rinse-off hair coloring/cleansing composition, comprising, or prepared by combining:
a) from about 0.1% to about 10% by weight of water-soluble dye materials;
b) from about 5% to about 50% by weight of surfactant materials comprising a member selected from the group consisting of anionic surfactant, amphoteric surfactant, and mixtures thereof;
c) from about 40% to about 94.9% by weight of water;
said water-soluble dye materials being concentrated in a dispersed phase of liquid emulsion droplets dispersed within a continuous aqueous phase; wherein said dye materials are concentrated within said droplets to the extent that the intensity of the color imparted by the dye materials to the droplets is greater than the intensity of the color imparted by the dye materials to the continuous aqueous phase.

2. A rinse-off hair coloring/cleansing composition according to claim 1, wherein the average size of said liquid emulsion droplets are between 0.05 μm to about 100 μm in diameter.

3. A rinse-off hair coloring/cleansing composition according to claim 1, wherein said surfactant materials are present at a concentration of from about 10% to about 20% by weight.

4. A rinse-off hair coloring/cleansing composition according to claim 1, wherein said water-soluble dye materials are present at a concentration of from about 0.2% to about 5% by weight.

5. A rinse-off hair coloring/cleansing composition according to claim 1, wherein said surfactant materials and said water-soluble dye materials interact to form the dispersed liquid phase in which said water-soluble dye materials are concentrated.

6. A rinse-off hair coloring/cleansing composition according to claim 1, wherein said anionic surfactant is selected from the group consisting of: organic sulfates, alkyl sulphates, alkaryl sulphates, ether sulfates, alkyl ether sulfates, lauryl ether sulfates, aryl alkyl sulphonates, alkyl sulphonates, alkaryl sulphonates, a-olefin sulfonates, organic phosphate esters, alkyl phosphates, alkyl taurates, alkyl isethionates, alkyl and alkenyl carboxylates, alkyl amidoether carboxylic acids, acylsarcosinates, dialkylsulphosuccinates, sulfonate salts, alkali salts of long-chain mono- and dialkyl phosphates, alkali salts of sulfosuccinic acid semiesters, fatty acid sarcosinates, fatty acid alkanolamide sulphosuccinates, and mixtures thereof.

7. A rinse-off hair coloring/cleansing composition according to claim 1, wherein said amphoteric surfactant is selected from the group consisting of: betaines, amidobetaines, sultaines, glycinates, propionates, amphoacetates, asparagine derivatives, and mixtures thereof.

8. A rinse-off hair coloring/cleansing composition according to claim 5, wherein said water-soluble dye materials consist of direct dyes.

9. A rinse-off hair coloring/cleansing composition according to claim 8, wherein said direct dyes are cationic.

10. A rinse-off hair coloring/cleansing composition according to claim 1 which also contains a cationic polymeric deposition aid which forms a coacervate with said surfactant materials to aid deposition of the dispersed dye phase.

11. A rinse-off hair coloring/cleansing composition according to claim 1, wherein said surfactant materials form a separated aqueous phase and said water-soluble dye preferentially partitions into this separated aqueous phase and thereby forming droplets, such that, microscopically, the color is more intense in dispersed droplets than in a continuous phase.

12. A rinse-off hair coloring/cleansing composition according to claim 11 further comprising non-surfactant electrolytes which aid in creation of the separated phase.

13. A rinse-off hair coloring/cleansing composition according to claim 12, wherein said surfactant materials consist of an anionic and/or amphoteric surfactant and form a separate aqueous phase by interacting and combining with a cationic polymer.

14. A rinse-off hair coloring/cleansing composition according to claim 11, wherein said surfactant materials consist of an anionic and/or amphoteric surfactant and form a separate aqueous phase by interacting and combining with a cationic polymer.

15. A rinse-off hair coloring/cleansing composition according to claim 14, wherein said separated aqueous phase is a liquid crystal phase.

16. A rinse-off hair coloring/cleansing composition according to claim 14, wherein said cationic polymer is comprises from about 0.1% to about 10% by weight of the composition.

17. A rinse-off hair coloring/cleansing composition according to claim 16, wherein said cationic polymer is selected from the group consisting of: cationic polymer of hydroxyethyl cellulose reacted with a trimethyl ammonium substituted epoxide, polymer of dimethyl diallyl ammonium chloride, guar hydroxypropyltrimonium chloride, polymethacrylamido-propyl trimonium chloride, and mixtures thereof.

18. A rinse-off hair coloring/cleansing composition according to claim 14, wherein said anionic surfactant is selected from the group comprising of: organic sulfates, alkyl sulphates, alkaryl sulphates, ether sulfates, alkyl ether sulfates, lauryl ether sulfates, aryl alkyl sulphonates, alkyl sulphonates, alkaryl sulphonates, α-olefin sulfonates, organic phosphate esters, alkyl phosphates, alkyl taurates, alkyl isethionates, alkyl and alkenyl carboxylates, alkyl amidoether carboxylic acids, acylsarcosinates, sulfonate salts, alkali salts of long-chain mono- and dialkyl phosphates, alkali salts of sulfosuccinic acid semiesters, dialkylsulphosuccinates fatty acid sarcosinates, fatty acid alkanolamide sulphosuccinates, and mixtures thereof.

19. A rinse-off hair coloring/cleansing composition according to claim 14, wherein said amphoteric surfactant is selected from the group comprising of: betaines, sultaines, amphoacetates, glycinates, propionates, asparagine derivatives, and mixtures thereof.

20. A rinse-off hair coloring/cleansing composition according to claim 1 further comprising silicones.

21. A rinse-off hair coloring/cleansing composition according to claim 1 additionally comprising a member selected from the group consisting of fatty alcohols, styling agents, perfumes, preservatives, anti-static agents, lather boosters, anti-dandruff agents, viscosity adjusting agents, thickeners, pH adjusting agents, anti-microbial agents, anti-oxidants, diluents, pearlescent aids, scalp senates, topical anesthetics, proteins, skin active agents, sunscreening agents, humectants, vitamins, pediculocides, and mixtures thereof.

22. A method of coloring and cleansing hair, comprising:
a) wetting said hair with water;
b) applying to said hair an effective amount of a coloring/cleansing composition according to claim 1; and
c) rinsing said coloring/cleansing composition from said hair using water.
